(19) United States
(12) Patent Application Publication
(10) Pub. No.: US 2010/0042202 A1
(43) Pub. Date: Feb. 18, 2010

(54) COMPOSITE STENT HAVING MULTI-AXIAL FLEXIBILITY

(76) Inventors: Kamal RAMZIPOOR, Fremont, CA (US); Richard J. SAUNDERS, Redwood City, CA (US)

Correspondence Address: LEVINE BAGADE HAN LLP, 2400 GENG ROAD, SUITE 120, PALO ALTO, CA, 94303, US

(21) Appl. No.: 12/541,095

(22) Filed: Aug. 13, 2009

Related U.S. Application Data
(60) Provisional application No. 61/088,433, filed on Aug. 13, 2008.

(51) Int. Cl. A61F 2/06; B05D 3/00; B32B 1/08

(52) U.S. Cl. 623/1.15; 623/1.44; 427/2.25; 428/36.9

(57) ABSTRACT

Composite stent structures having multi-axial flexibility are described where the composite stent may have one or more layers of bioabsorbable polymers fabricated with the desired characteristics for implantation within a vessel. A number of individual ring structures separated from one another may be encased between a base polymeric layer and an overlaid polymeric layer such that the rings are coupled to one another via elastomeric segments which enable the composite stent to flex axially and rotationally along with the vessel. Each layer may have a characteristic that individually provides a certain aspect of mechanical behavior to the composite stent such that the aggregate layers form a composite polymeric stent structure capable of withstanding complex, multi-axial loading conditions imparted by an anatomical environment such as the SFA.

### CROSS-REFERENCE TO RELATED APPLICATIONS

This application claims the benefit of priority to U.S. Prov. Pat. App. 61/088,433 filed Aug. 13, 2008, which is incorporated herein by reference in its entirety.

### FIELD OF THE INVENTION

The present invention relates generally to composite prostheses which are implantable within a patient. More particularly, the present invention relates to implantable tubular prostheses, such stents, which utilizes a composite structure having various geometries suitable for implantation within a patient.

### BACKGROUND OF THE INVENTION

In recent years there has been growing interest in the use of artificial materials, particularly materials formed from polymers, for use in implantable devices that come into contact with bodily tissues or fluids particularly blood. Some examples of such devices are artificial heart valves, stents, and vascular prosthesis. Some medical devices such as implantable stents which are fabricated from a metal have been problematic in fracturing or failing after implantation. Moreover, certain other implantable devices made from polymers have exhibited problems such as increased wall thickness to prevent or inhibit fracture or failure. However, stents having reduced wall thickness are desirable particularly for treating arterial diseases.

Because many polymeric implants such as stents are fabricated through processes such as extrusion or injection molding, such methods typically begin the process by starting with an inherently weak material. In the example of a polymeric stent, the resulting stent may have imprecise geometric tolerances as well as reduced wall thicknesses which may make these stents susceptible to brittle fracture.

A stent which is susceptible to brittle fracture is generally undesirable because of its limited ability to collapse for intravascular delivery as well as its limited ability to expand for placement or positioning within a vessel. Moreover, such polymeric stents also exhibit a reduced level of strength. Brittle fracture is particularly problematic in stents as placement of a stent onto a delivery balloon or within a delivery sheath imparts a substantial amount of compressive force in the material comprising the stent. A stent made of a brittle material may crack or have a very limited ability to collapse or expand without failure. Thus, a certain degree of malleability is desirable for a stent to expand, deform, and maintain its position securely within the vessel.

Certain indications, such as peripheral arterial disease, affects millions of people where the superficial femoral artery (SFA) is commonly involved. Stenosis or occlusion of the SFA is a common cause of many symptoms such as claudication and is often part of critical limb ischemia. Although interventional therapy for SFA diseases using Nitinol stents is increasing, the SFA poses particular problems with respect to stent implantation because the SFA typically elongates and foreshortens with movement, can be externally compressed, and is subject to flexion. Limitations of existing stents include, e.g., insufficient radial strength to withstand elastic recoil and external compression, kinking, and fracture.

Because of such limitations, stent fractures have been reported to occur in the iliac, popliteal, subclavian, pulmonary, renal, and coronary arteries. However, it is suspected that these fractures may occur at a higher rate in the SFA than the other locations. For example, because the SFA can undergo dramatic non-pulsatile deformations (e.g., axial compression and extension, radial compression bending, torsion, etc.) such as during hip and knee flexion causing significant SFA shortening and elongation and because the SFA has a tendency to develop long, diffuse, disease states with calcification requiring the use of multiple overlapping stents, stent placement, maintenance, and patency is difficult. Moreover, overlapping of adjacent stents cause metal-to-metal stress points that may initiate a stent fracture.

Accordingly, there is a need for an implantable stent that is capable of withstanding dynamic loading conditions of the SFA or similar environments.

### SUMMARY OF THE INVENTION

When a stent is placed into a vessel (particularly vessels such as the superficial femoral artery (SFA), iliac, popliteal, subclavian, pulmonary, renal, coronary arteries, etc.), the stent's ability to bend and compress is reduced. Moreover, such vessels typically undergo a great range of motion requiring stents implanted within these vessels to have an axial flexibility which allows for its compliance with the arterial movement without impeding or altering the physiological axial compression and bending normally found with positional changes.

A composite stent structure having one or more layers of bioabsorbable polymers may be fabricated with the desired characteristics for implantation within these vessels. Each layer may have a characteristic that individually provides a certain aspect of mechanical behavior to the stent such that the aggregate layers form a composite polymeric stent structure capable of withstanding complex, multi-axial loading conditions imparted by an anatomical environment such as the SFA.

Generally, a tubular substrate may be constructed by positioning one or more high-strength bioabsorbable polymeric ring structures spaced apart from one another along a longitudinal axis. The ring structures may be connected to one another by one or more layers of polymeric substrates, such as bioabsorbable polymers which are also elastomeric. Such a structure is made of several layers of bioabsorbable polymers with each layer having a specific property that positively affects certain aspect of mechanical behavior of the stent and all layers collectively as a composite polymeric material create a structure capable of withstanding complex, multi axial loading conditions of an anatomical environment such as SFA.

A number of casting processes may be utilized to develop substrates, e.g., cylindrically shaped substrates, having a relatively high level of geometric precision and mechanical strength for forming the ring structures. These polymeric substrates can then be machined using any number of processes (e.g., high-speed laser sources, mechanical machining, etc.) to create devices such as stents having a variety of geometries for implantation within a patient, such as the peripheral or coronary vasculature, etc.

An example of such a casting process is to utilize a dip-coating process. The utilization of dip-coating to create a polymeric substrate having such desirable characteristics results in substrates which are able to retain the inherent properties of the starting materials. This in turn results in substrates having a relatively high radial strength which is retained through any additional manufacturing processes for implantation. Additionally, dip-coating the polymeric substrate also allows for the creation of substrates having multiple layers.

The molecular weight of a polymer is typically one of the factors in determining the mechanical behavior of the polymer. With an increase in the molecular weight of a polymer, there is generally a transition from brittle to ductile failure. A mandrel may be utilized to cast or dip-coat the polymeric substrate. Further examples of high-strength bioabsorbable polymeric substrates formed via dip-coating processes are described in further detail in U.S. patent application Ser. No. 12/143,659 filed Jun. 20, 2008, which is incorporated herein by reference in its entirety.

The substrate may also be machined, e.g., using laser ablation processes, to produce stents with suitable geometries for particular applications. The composite stent structure may have a relatively high radial strength as provided by the polymeric ring structures while the polymeric portions extending between the adjacent ring structures may allow for elastic compression and extension of the stent structure axially as well as torsionally when axial and rotational stresses are imparted by ambulation and positional changes from the vessel upon the stent structure.

### BRIEF DESCRIPTION OF THE DRAWINGS

FIG. 1A shows an example of a polymeric substrate having one or more layers formed by dip coating processes creating a substrate having a relatively high radial strength and ductility.

FIG. 1B shows an example the formed polymeric substrate cut into a number of circular ring-like structures.

FIG. 2 shows another polymeric layer, e.g., elastomeric in nature, formed as a base substrate.

FIG. 3A illustrates an example of how the circular ring-like structures may be positioned or fitted upon the base substrate to form an intermediate layer of a composite stent structure.

FIG. 3B shows the composite structure formed with an additional polymeric layer, e.g., elastomeric in nature, overlaid atop the base substrate and ring structures.

FIG. 4 shows an example of another variation of the composite structure where the ring structures may be patterned to form a scaffold structure.

FIG. 5 shows another variation where the ring structures may be alternated between rings fabricated from different polymeric substrates.

FIG. 6 shows another variation where one or more terminal rings may be formed of a flexible ring structure for overlapping between adjacently deployed stents.

FIG. 7 shows another variation where each ring structure along the composite stent may be fabricated from polymeric substrates different from one another.

FIG. 8 shows another variation where the intermediate polymeric layer is formed as longitudinal strips rather than ring structures.

FIG. 9 shows yet another variation where the intermediate polymeric layer is formed as a helical structure between the base layer and overlaid layer.

FIG. 10A illustrates an example of adjacent composite stent structures deployed within a vessel with a gap or spacing between the stent structures.

FIG. 10B illustrates another example of adjacent composite stent structures deployed within a vessel with the terminal ends of the stents overlapped with one another.

FIG. 11 illustrates a side view of another variation where the terminal ring structures are configured to degrade at a relatively faster rate than the remaining ring structures.

FIGS. 12A and 12B illustrate side views of yet another variation where polymeric ring structures are positioned along a flexible base coat in a separate manufacturing operation.

FIGS. 13A and 13B illustrate partial cross-sectional side and end views, respectively, of a composite structure formed by sandwiching a high-strength polymeric material between two or more layers of a flexible polymer to provide for greater flexibility under radial stress while retaining relatively high strength.

FIGS. 14A and 14B illustrate perspective views, respectively, of a polymeric substrate which may be machined to form one or more reduced segments along the length of the substrate.

FIGS. 14C and 14D illustrate perspective and partial cross-sectional perspective views, respectively, of a machined substrate further coated by one or more polymeric layers.

FIG. 15 shows an example of a stent or scaffold which may be formed from the polymeric substrate having various portions of the stent, e.g., such as the struts, fabricated from the thickened segments of the substrate.

FIG. 16 shows another example of a stent or scaffold which may be alternatively formed from the polymeric substrate such that alternating circumferential segments are fabricated from either thickened or thinned segments of the substrate.

FIGS. 17A and 17B illustrate a polymeric substrate which has been machined to form ring segments connected via connecting members placed upon a mandrel, respectively.

FIGS. 18A and 18B illustrate the machined substrate coated by one or more polymeric layers and a partial cross-sectional side view, respectively.

FIG. 19 shows an example of a stent or scaffold which may be formed from the polymeric substrate having portions of the stent, e.g., struts, formed from the coated polymeric layers.

FIG. 20 shows another example of a stent or scaffold which may be formed from the polymeric substrate to have alternating circumferential segments fabricated from either thickened or thinned segments of the substrate.

### DETAILED DESCRIPTION OF THE INVENTION

When a stent is placed into a vessel (particularly vessels such as the superficial femoral artery (SFA), iliac, popliteal, subclavian, pulmonary, renal, coronary arteries, etc.), the stent's ability to bend and compress is reduced. Moreover, such vessels typically undergo a great range of motion requiring stents implanted within these vessels to have an axial flexibility which allows for its compliance with the arterial movement without impeding or altering the physiological axial compression and bending normally found with positional changes.

A composite stent structure having one or more layers of bioabsorbable polymers may be fabricated with the desired characteristics for implantation within these vessels. Each layer may have a characteristic that individually provides a certain aspect of mechanical behavior to the stent such that the aggregate layers form a composite polymeric stent structure capable of withstanding complex, multi-axial loading conditions imparted by an anatomical environment such as the SFA.

Generally, a tubular substrate may be constructed by positioning one or more high-strength bioabsorbable polymeric ring structures spaced apart from one another along a longitudinal axis. The ring structures may be connected to one another by one or more layers of polymeric substrates, such as bioabsorbable polymers which are also elastomeric. The substrate may also be machined, e.g., using laser ablation processes, to produce stents with suitable geometries for particular applications. The composite stent structure may have a relatively high radial strength as provided by the polymeric ring structures while the polymeric portions extending between the adjacent ring structures may allow for elastic compression and extension of the stent structure axially as well as torsionally when axial and rotational stresses are imparted by ambulation and positional changes from the vessel upon the stent structure.

In manufacturing the polymeric ring structures from polymeric materials such as biocompatible and/or biodegradable polymers (e.g., polylactic acid (PLLA) 2.4, PLLA 4.3, PLLA 8.4, PLA, PLGA, etc.), a number of casting processes may be utilized to develop substrates, e.g., cylindrically shaped substrates, having a relatively high level of geometric precision and mechanical strength. A high-strength tubular material which exhibits a relatively high degree of ductility may be fabricated utilizing such polymers having a relatively high molecular weight These polymeric substrates can then be machined using any number of processes (e.g., high-speed laser sources, mechanical machining, etc.).

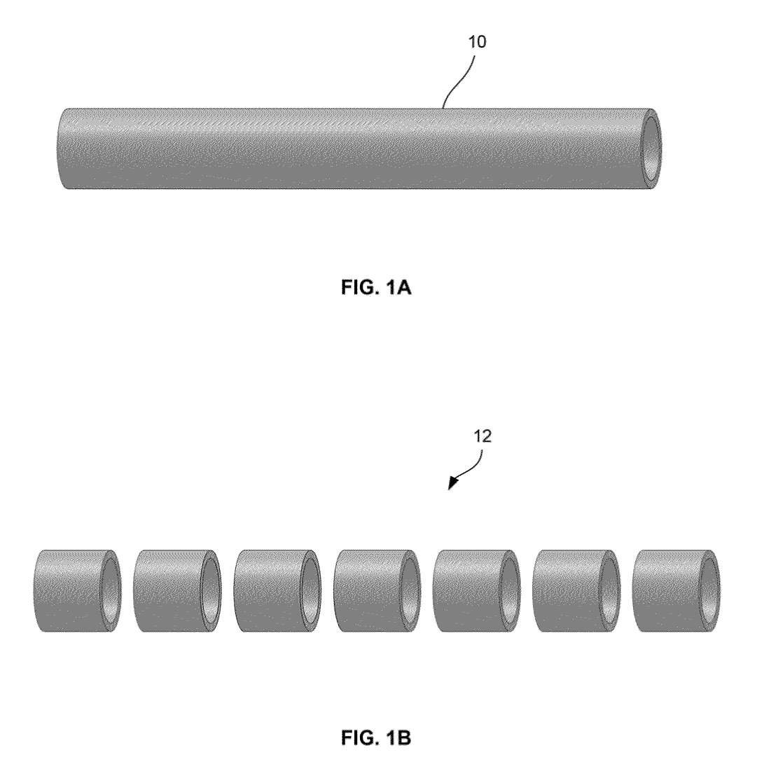

An example of such a casting process is to utilize a dip-coating process. The utilization of dip-coating to create a polymeric substrate 10 having such desirable characteristics results in substrates 10 which are able to retain the inherent properties of the starting materials, as illustrated in FIG. 1A. This in turn results in substrates 10 having a relatively high radial strength which is mostly retained through any additional manufacturing processes for implantation. Additionally, dip-coating the polymeric substrate 10 also allows for the creation of substrates having multiple layers. The multiple layers may be formed from the same or similar materials or they may be varied to include any number of additional agents, such as one or more drugs for treatment of the vessel, as described in further detail below. Moreover, the variability of utilizing multiple layers for the substrate may allow one to control other parameters, conditions, or ranges between individual layers such as varying the degradation rate between layers while maintaining the intrinsic molecular weight and mechanical strength of the polymer at a high level with minimal degradation of the starting materials.

Because of the retention of molecular weight and mechanical strength of the starting materials via the casting or dip-coating process, polymeric substrates 10 may be formed which enable the fabrication of devices such as stents with reduced wall thickness which is highly desirable for the treatment of arterial diseases. Furthermore these processes may produce structures having precise geometric tolerances with respect to wall thicknesses, concentricity, diameter, etc.

One mechanical property in particular which is generally problematic with, e.g., polymeric stents formed from polymeric substrates, is failure via brittle fracture of the device when placed under stress within the patient body. It is generally desirable for polymeric stents to exhibit ductile failure under an applied load rather via brittle failure, especially during delivery and deployment of a polymeric stent from an inflation balloon or constraining sheath.

Further examples of high-strength bioabsorbable polymeric substrates formed via dip-coating processes are described in further detail in U.S. patent application Ser. No. 12/143,659 filed Jun. 20, 2008, which is incorporated herein by reference in its entirety. Such dip-coating methods may be utilized to create polymeric substrates such as substrate 10, which may then be cut into a plurality of polymeric ring structures 12, as shown in FIG. 1B. These ring structures may have a width which varies depending upon the application and vessel and may range generally from 1 mm to 10 mm in width. Moreover, because the initial polymeric substrate 10 is formed upon a mandrel, substrate 10 and the resulting ring structures 12 may be formed to have an initial diameter ranging generally from 2 mm to 10 mm.

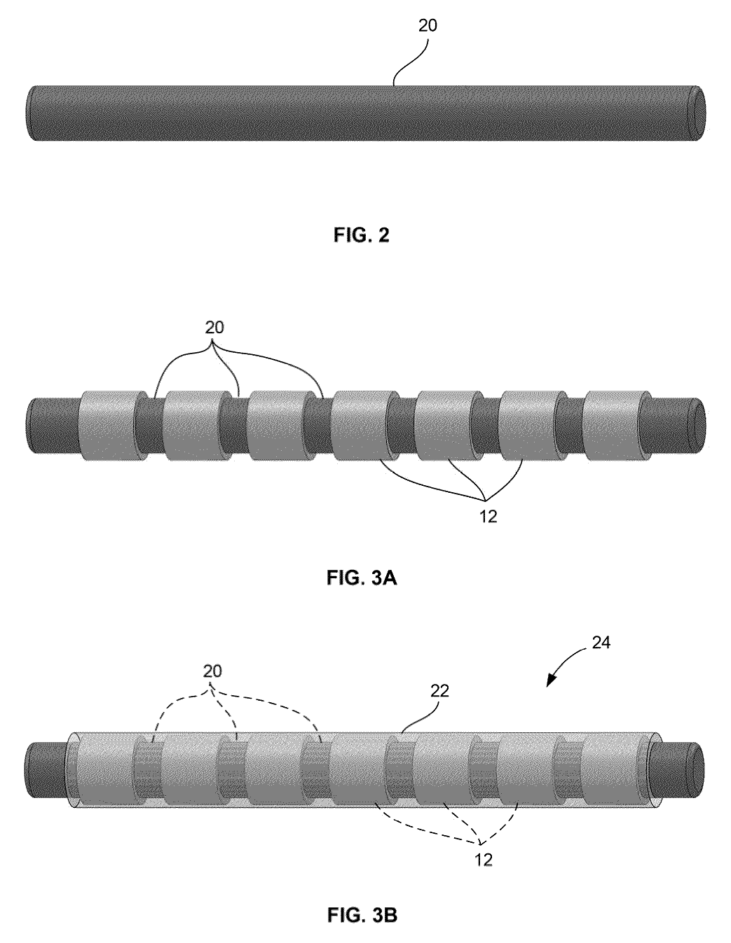

Another polymeric substrate may also be formed, e.g., also via dip-coating, upon a mandrel to form a base polymeric substrate 20, as shown in FIG. 2. The base substrate 20 may be formed of, e.g., an elastomeric bioabsorbable polymer resin such as polycaprolactone (PCL), trimethylene carbonate (TMC), etc., which is dissolved in a compatible solvent such as dichloromethane (DCM). The polymeric solution may be poured into a container and placed under a dipping machine in an inert environment. A mandrel that is attached to the dipping machine immerses into the solution and creates the base layer of the composite stent structure. Once formed, the resulting polymeric substrate 20 may have an initial diameter, e.g., ranging generally from 2 mm to 10 mm, defined by the mandrel which is similar to the diameter of the ring structures 12. The substrate 20 may be formed to have an initial length ranging from 5 mm to 500 mm. The substrate 20 may be left upon the mandrel or removed and placed upon another mandrel.

In either case, the ring structures 12 may be positioned upon the base polymeric substrate 20, as illustrated in FIG. 3A, at uniform intervals or at predetermined non-uniform distances from one another. The spacing between the ring structures 12 may be determined in part by the degree of flexibility desired of the resulting composite stent structure where the closer adjacent ring structures 12 are positioned relative to one another, the lesser resulting overall stent flexibility. Additionally, ring structures 12 may be positioned relatively closer to one another along a first portion of the composite stent and relatively farther from one another along a second portion of the stent. In one example, the ring structures 12 may be positioned at a uniform distance of 1 mm to 10 mm from one another.

If the ring structures 12 are formed to have a diameter which is slightly larger than a diameter of the base polymeric substrate 20, the ring structures 12 may be compressed to reduce their diameters such that the ring structures 12 are overlaid directly upon the outer surface of the substrate 20. In use, the ring structures 12 may be compressed to a second smaller diameter for delivery through the vasculature of a patient to a region to be treated. When deployed, the ring structures 12 (as well as the base substrate 20 and overlaid substrate 22) may be expanded back to their initial diameter or to a diameter less than the initial diameter.

The ring and substrate structure may then be immersed again in the same or different polymeric solution as base polymeric substrate 20 to form an additional polymeric substrate 22 overlaid upon the base substrate 20 and ring structures 12 to form the composite stent structure 24, as illustrated in FIG. 3B. The ring structures 12 may be encapsulated or otherwise encased entirely between the base substrate 20 and the overlaid substrate 22 such that the ring structures 12 are connected or otherwise attached to one another entirely via the elastomeric sections.

Additionally, either or both of the ring structures 12 and base or overlaid substrate layers 20, 22 may be configured to retain and deliver or elute any number of agents, such as antiproliferative, antirestenotic pharmaceuticals, etc.

Because the elastomeric polymer substrate couples the ring structures 12 to one another rather than an integrated structural connecting member between the ring structures themselves, the ring structures 12 may be adjustable along an axial or radially direction independently of one another allowing for any number of configurations and adjustments of the stent structure 24 for conforming within and bending with a vessel which other coated stent structures are unable to achieve.

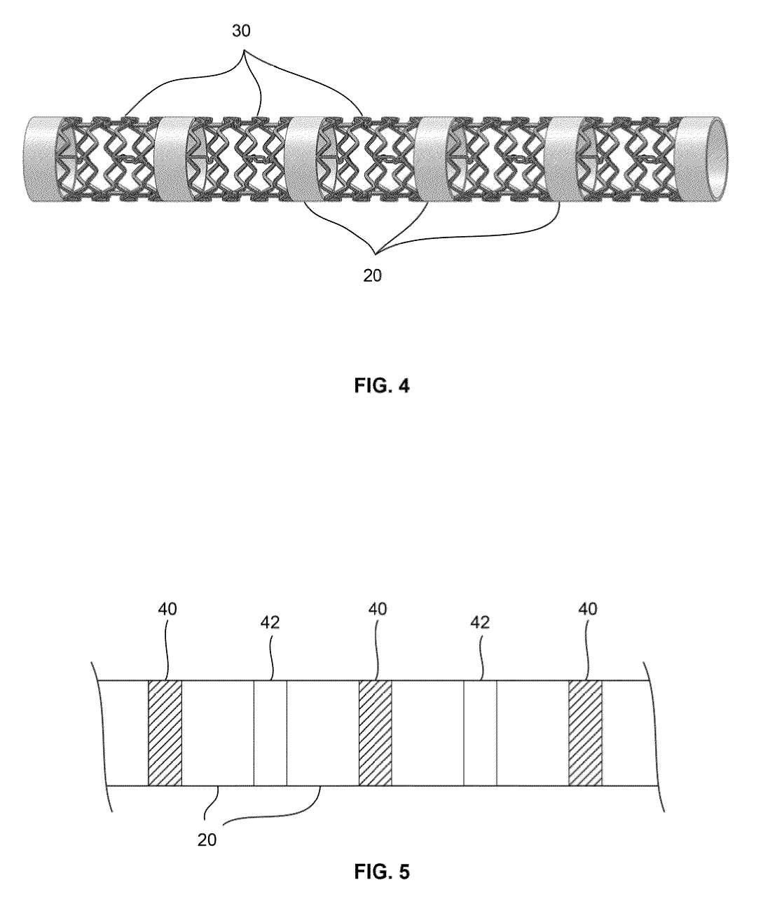

This resulting stent structure 24 may be removed from the mandrel and machined to length, if necessary, and additional post-processing may be performed upon the stent as well. For instance, the stent structure 24 may have one or more of the ring structures machined into patterned polymeric rings 30 such as expandable scaffold structures, e.g., by laser machining, as illustrated in FIG. 4. In machining the stent structure, the process of removing material from the polymeric rings 30 may at least partially expose portions of the polymeric rings 30 to the environment. For example, the inner surfaces and the outer surfaces of the polymeric rings 30 may remain coated or covered by both respective base and overlaid substrate layers 20, 22 while side surfaces of the rings 30 may become exposed by removal of the substrate layers as well as portions of the ring material as the stent structure is machined. These exposed surfaces may be re-coated, if desired, or left exposed to the environment.

The polymeric ring structures 12 utilized in the composite stent structure 24 may be fabricated from a common substrate and common polymers. However, in other variations, the ring structures forming the stent 24 may be fabricated from different substrates having different material characteristics. FIG. 5 illustrates an example where a first set of polymeric rings 40 may be positioned in an alternating pattern with a second set of polymeric rings 42 along the base substrate 20. In this and other examples, the overlaid polymeric substrate 22 may be omitted from the figures merely for clarity.

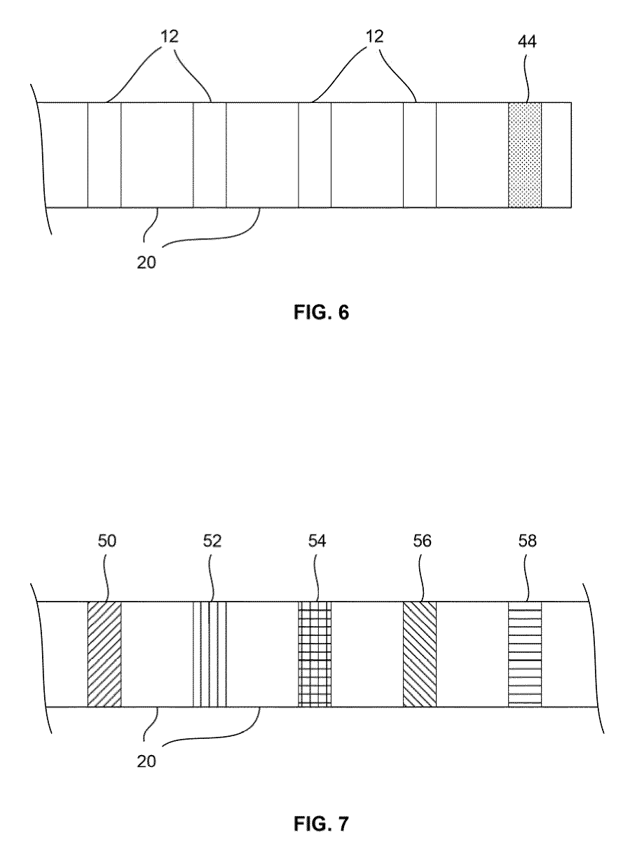

Another variation is illustrated in FIG. 6, which shows an example where a first set of polymeric ring structures 12 may be positioned along the stent with a flexible polymeric ring 44 fabricated to be relatively more flexible than the remaining ring structures 12 positioned along a terminal end of the stent structure.

Yet another example is illustrated in FIG. 7 where each of the ring structures may be fabricated from different substrates and polymers. For example, a stent structure may be fabricated to have a first polymeric ring 50, a second polymeric ring 52, a third polymeric ring 54, a fourth polymeric ring 56, a fifth polymeric ring 58, and so on to form the composite stent structure. An example of use may include a composite stent structure for placement within a tapered or diametrically expanding vessel where each subsequent ring structure may be fabricated to be more radially expandable than an adjacent ring structure, e.g., where the first polymeric ring 50 may be radially expandable to a first diameter, second polymeric ring 52 is radially expandable to a second diameter larger than the first diameter, third polymeric ring 54 may be radially expandable to a third diameter larger than the second diameter, and so on. This is intended to be exemplary and other examples are, of course, intended to be within the scope of this disclosure.

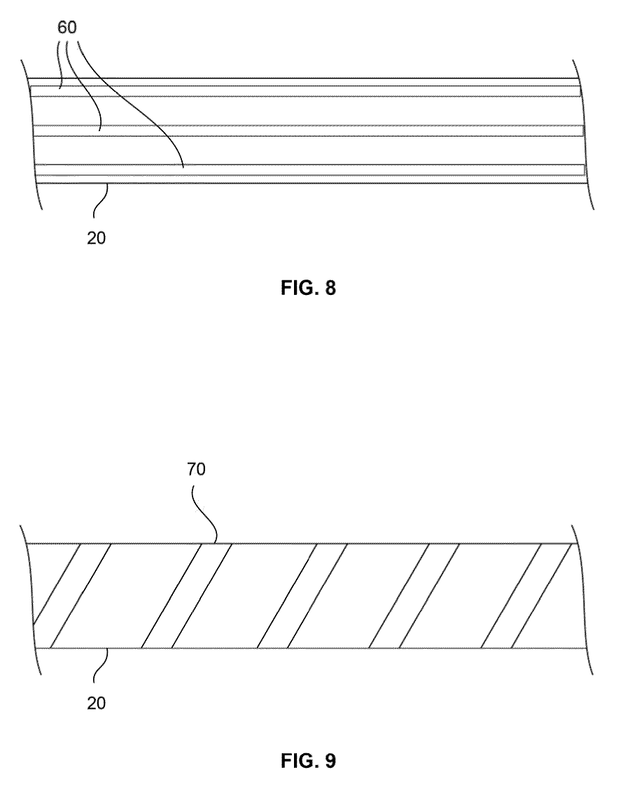

Yet another variation is shown in FIG. 8, which illustrates longitudinally-oriented polymeric strips 60 rather than ring structures positioned along the base substrate 20. In this example, such a composite stent structure may be configured to allow for greater flexibility under radial stresses. Another example is illustrated in FIG. 9 which shows a helically-oriented polymeric member 70 which may be positioned along base substrate 20.

As described in U.S. patent application Ser. No. 12/143,659 incorporated hereinabove, the polymeric substrate utilized to form the ring structures may be heat treated at, near, or above the glass transition temperature T_{g} of the substrate to set an initial diameter and the substrate may then be processed to produce the ring structures having a corresponding initial diameter. The resulting composite stent structure 24 may be reduced from its initial diameter to a second delivery diameter which is less than the initial diameter such that the composite stent structure 24 may be positioned upon, e.g., an inflation balloon of a delivery catheter. The composite stent structure 24 at its reduced diameter may be self-constrained such that the stent remains in its reduced diameter without the need for an outer sheath, although a sheath may be optionally utilized. Additionally, the composite stent structure 24 may be reduced from its initial diameter to its delivery diameter without cracking or material failure.

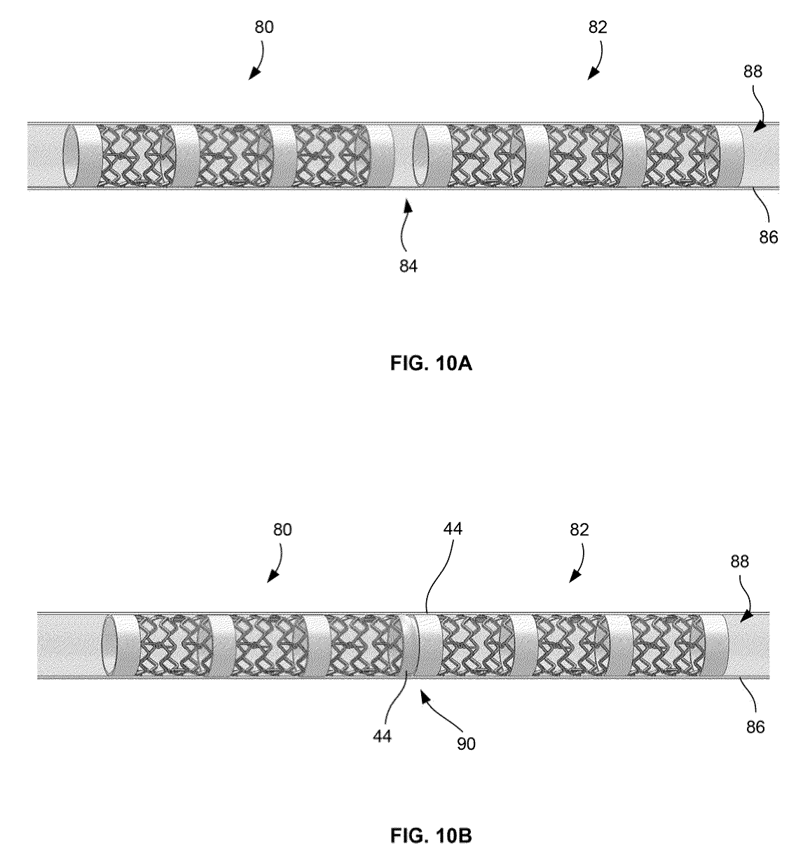

With the composite stent structure positioned upon a delivery catheter, the stent may be advanced intravascularly within the lumen 88 of a vessel 86 until the delivery site is reached. The inflation balloon may be inflated to expand a diameter of composite stent structure into contact against the vessel interior, e.g., to an intermediate diameter, which is less than the stent's initial diameter yet larger than the delivery diameter. The composite stent structure may be expanded to this intermediate diameter without any cracking or failure because of the inherent material characteristics, as shown in FIG. 10A. Moreover, expansion to the intermediate diameter may allow for the composite stent structure to securely contact the vessel wall while allowing for the withdrawal of the delivery catheter.

Once the composite stent structure has been expanded to some intermediate diameter and secured against the vessel wall 86, composite stent structure 24 may be allowed to then self-expand further over a period of time into further contact with the vessel wall such that composite stent structure 24 conforms securely to the tissue. This self-expansion feature ultimately allows for the composite stent structure 24 to expand back to its initial diameter which had been heat set in the ring structures or until the composite stent structure 24 has fully self-expanded within the confines of the vessel lumen 88. In yet another variation, the composite stent structure 24 may be expanded directly to its final diameter, e.g., by balloon inflation, without having to reach an intermediate diameter and subsequent self-expansion.

In the example illustrated, a first composite stent 80 is shown deployed within vessel lumen 88 adjacent to a second composite stent 82 with spacing 84 between the stents. Additional stent structures may be deployed as well depending upon the length of the lesion to be stented. FIG. 10B illustrates another example where adjacent composite stents 80, 82 are deployed within vessel lumen 88 with their terminal ends overlapping one another along overlapped portion 90. As the SFA tends to develop long, diffuse lesions with calcification, multiple stents may be deployed with overlapping ends. However, as this overlapping may cause regions or locations of increased stress that can initiate fracturing along the stent and leading to potential stent failure and closure of the vessel, the terminal ring structures of both overlapped composite stents 80, 82 may be fabricated from an elastomeric polymer allowing for the overlap to occur along these segments. Such overlapping would not significantly compromise axial flexibility and the composite stents may continue its compliance with the arterial movement.

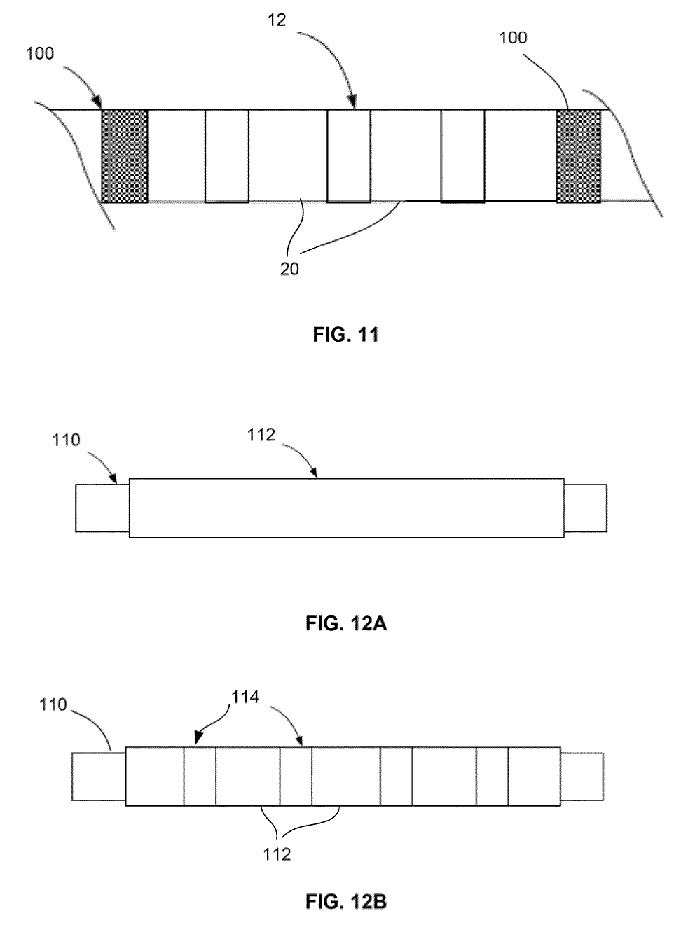

Another variation which facilitates the overlapping of adjacent stents is shown in the side view of FIG. 11. The overlaid substrate has been omitted for clarity only and may be included as a layer positioned atop the base substrate 20 as well as the polymeric rings, as previously described. As illustrated, the polymeric ring structures 12 may include terminal polymeric rings 100 which are fabricated to degrade at a relatively faster rate than the remaining ring structures 12 positioned between these terminal rings 100. Such a composite stent structure may allow for the optimal overlapping of multiple stents along the length of a blood vessel.

Yet another variation is shown in the side views of FIGS. 12A and 12B which illustrate a mandrel 110 that is provided with a flexible polymeric base substrate 112 placed or formed thereon. A set of polymeric ring structures 114 may be positioned along the longitudinal axis of the flexible base coat 112 in a separate manufacturing operation.

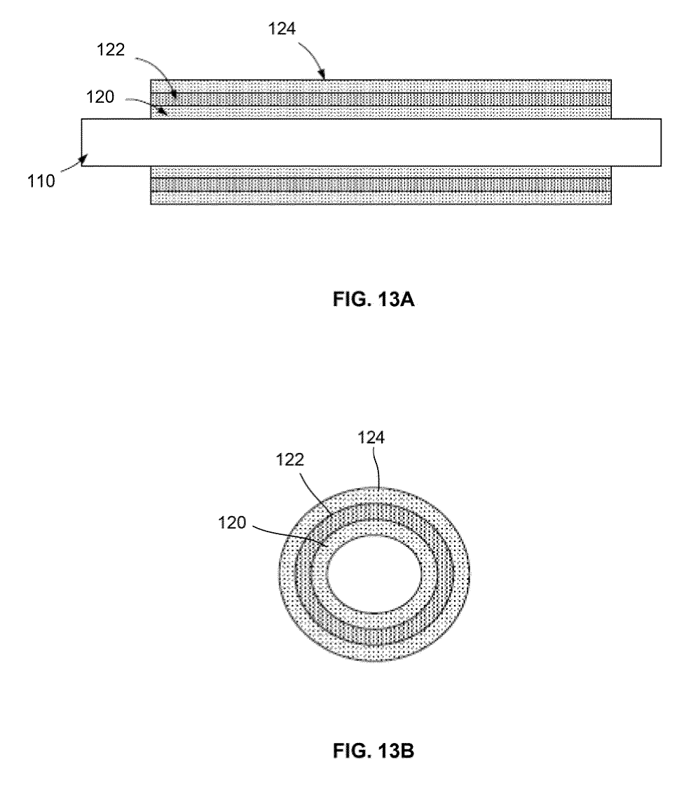

Another variation is illustrated in the partial cross-sectional side and end views, respectively, of FIGS. 13A and 13B. In this example, a composite structure may be provided by layering multiple coatings. For instance, a middle layer 122 may be made of a high strength polymeric material such as PLLA (polylactic acid) that is sandwiched between two or more layers 120, 124 of a flexible polymer such as PCL (polycaprolactone). Such a composite stent structure may be configured to allow for greater flexibility under radial stresses while retaining relatively high strength provided by the PLLA layer 122.

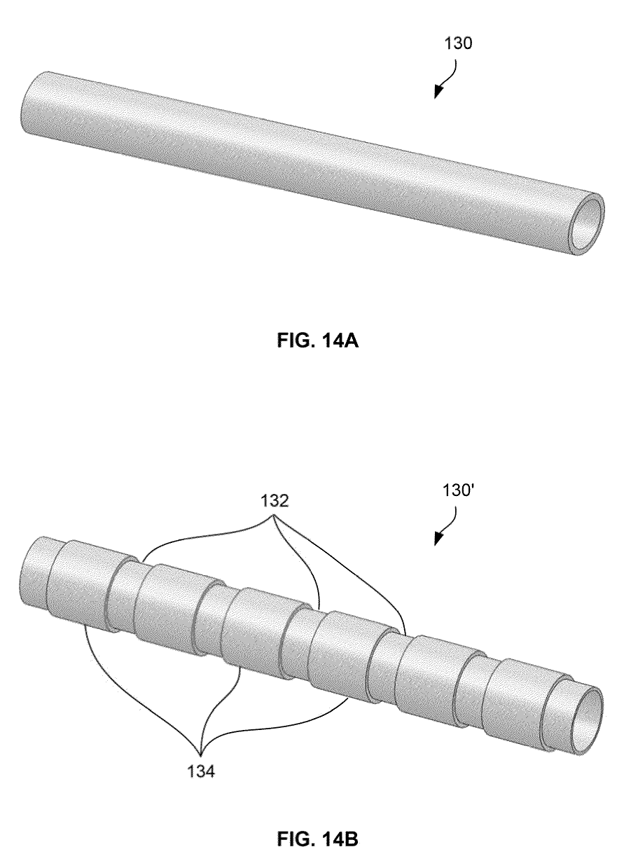

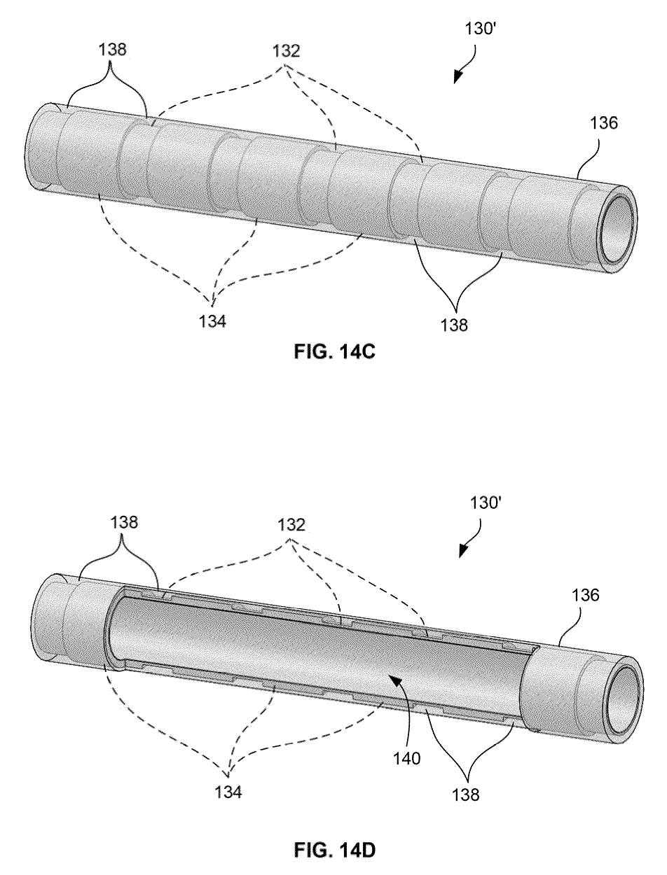

In yet other alternative variations for forming composite structures, a bioabsorbable polymeric substrate 130, e.g., initially formed by the dip-coating process as previously described, may be formed into a tubular substrate as shown in the perspective view of FIG. 14A. Substrate 130 may be further processed, such as by machining, to form a machined substrate 130′, as shown in the perspective view of FIG. 14B, having one or more reduced segments 132 which are reduced in diameter alternating with the relatively thicker segments 134 which may be reduced in diameter to a lesser degree or uncut altogether. The number of reduced segments 132 and the spacing between may be uniform or varied depending upon the desired resulting stent or scaffold and the reduction in diameter of these segments 132 may also be varied as well. In one example, for a given initial diameter of 2 to 12 mm of substrate 130, segments 132 may be reduced in diameter by, e.g., 1.85 to 11.85 mm. Moreover, although the example shown in FIG. 14B shows seven reduced segments 132 between thicker segments 134, this number may be varied depending upon the desired resulting lengths of segments 132, e.g., ranging from 0.5 mm to 3 mm in length.

In forming the substrate to have a variable wall thickness as illustrated, laser machining (profiling) of the outer diameter may be utilized. The integrity and material properties of the substrate material is desirably maintained during this process of selectively removing material in order to achieve the desired profile. An ultra-short pulse femto-second type laser may be used to selectively remove the material from the reduced segments 132 by taking advantage, e.g., of multi-photon absorption, such that the laser removes the material without modifying the material integrity. Thus, the mechanical properties and molecular structure of the bio-absorbable substrate 130 may be unaffected during this machining process.

Some of the variables in utilizing such a laser for this particular application may include, e.g., laser power level, laser pulse frequency, energy profile of the beam, beam diameter, lens focal length, focal position relative to the substrate surface, speed of the substrate/beam relative to the substrate, and any gas jet/shield either coaxial or tangential to the material, etc. By adjusting some or all of these variables, a multi-level profile can be readily produced. In one example, increasing or decreasing the rotational speed of the substrate relative to the laser during processing will vary the depth of penetration. This in combination with a translation rate of the substrate relative to the laser can also be varied to produce a relatively sharp edge in the relief area or a smooth tapered transition between each of the adjacent segments. Varying both parameters along the longitudinal axis of the substrate 130 can produce a continuously variable profile from which a stent pattern can be cut, as further described below.

The laser system may comprise an ultra-short pulse width laser operating in the femto-second pulse region, e.g., 100 to 500 fs typical pulse width, and a wavelength, λ, e.g., in the near to mid-IR range (750 to 1600 nm typical λ). The pulse frequency of these lasers can range from single pulse to kilo-hertz (1 to 10 kHz typical). The beam energy profile can be TEMoo to a high order mode (TEMoo is typical, but not necessary). The beam delivery system may comprise a beam bender, vertical mounted monocular viewing/laser beam focusing head, focusing lens and coaxial gas jet assembly. A laser system may also include a linear stage having a horizontally mounted rotary stage with a collet clamping system mounted below the focusing/cutting head.

With the substrate tube 130 clamped by the rotary stage and held in a horizontal plane, the laser beam focusing head may be positioned perpendicular to the longitudinal axis of substrate 130. Moving the focus of the beam away from the outer diameter of the tubing, a non-penetrating channel can be machined in the substrate 130. Controlling the speed of rotation and/or linear translation of the tube under the beam, a channel can be machined along the substrate axis. Varying any one or all of the parameters (e.g., position, depth, taper, length, etc.) of machining can be controlled and positioned along the entire length of the substrate 130. The ability to profile the substrate 130 may provide a number of advantages in the flexibility of the resulting stent design and performance. For example, such profiling may improve the flexibility of the stent geometry and expansion capability in high stress areas, expose single or multiple layers to enhance or expose drug delivery by placing non-penetrating holes into one or more particular drug-infused layer(s) of the substrate 130 or by placing grooves or channels into these drug layer(s). Moreover, the ability to profile the substrate 130 may allow for a substrate having a variable profile which can be over-coated with the same or different polymer, as described herein.

Once machined substrate 130′ has been sufficiently processed, it may then be coated, e.g., via the dip-coating process as previously described, such that one or more additional elastomeric polymer layers are coated upon substrate 130′. The example shown in the perspective view of FIG. 14C illustrates machined substrate 130′ having at least one additional elastomeric polymer layer 136 coated thereupon; however, other variations may have more than one layer coated atop one another depending upon the desired characteristics of the resulting substrate. Additionally, each subsequent layer coated upon machined substrate 130′ may be of the same, similar, or different material from substrate 130′, e.g., polyethylene, polycarbonates, polyamides, polyesteramides, polyetheretherketone, polyacetals, polyketals, polyurethane, polyolefin, polyethylene terephthalate, polylactide, poly-L-lactide, poly-glycolide, poly(lactide-co-glycolide), polycaprolactone, caprolactones, polydioxanones, polyanhydrides, polyorthocarbonates, polyphosphazenes, chitin, chitosan, poly(amino acids), polyorthoesters, oligomers, homopolymers, methyl cerylate, methyl methacrylate, acryli acid, methacrylic acid, acrylamide, hydroxyethy acrylate, hydroxyethyl methacrylate, glyceryl scrylate, glyceryl methacrylate, methacrylamide, ethacrylamide, styrene, vinyl chloride, binaly pyrrolidone, polyvinyl alcohol, polycoprolactam, polylauryl lactam, polyjexamethylene adipamide, polyexamethylene dodecanediamide, trimethylene carbonate, poly(β-hydroxybutyrate), poly(g-ethyl glutamate), poly(DTH iminocarbonate), poly(bisphenol A iminocarbonate), polycyanoacrylate, polyphosphazene, methyl cerylate, methyl methacrylate, acryli acid, methacrylic acid, acrylamide, hydroxyethy acrylate, hydroxyethyl methacrylate, glyceryl scrylate, glyceryl methacrylate, methacrylamide, ethacrylamide, and copolymers, terpolymers and combinations and mixtures thereof, etc., again depending upon the desired resulting characteristics. The one or more polymeric layers 136 may be coated upon machined substrate 130′ such that the elastomeric polymer 136 forms within the reduced segments 132 as well as upon segments 134. The resulting coated layer 136 may range in thickness accordingly from, e.g., 50 μm to 500 μm, such that the layer 136 forms a uniform outer diameter along the length of substrate 130′. As shown in the partial cross-sectional perspective view of FIG. 14D, the thickened elastomeric polymer segments 138 formed along reduced segments 132 may be seen along substrate 130′ with substrate lumen 140 defined therethrough.

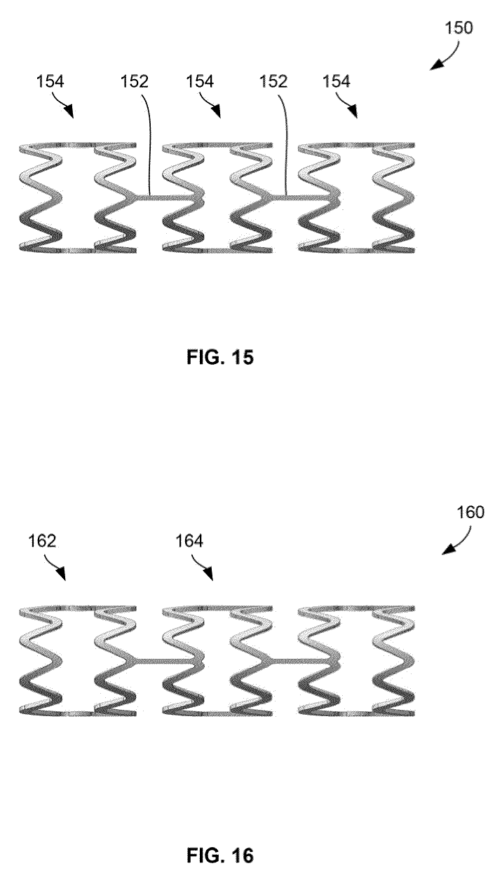

With machined substrate 130′ coated with the one or more polymeric layers 136, the entire formed substrate may then be processed, e.g., machined, laser-machined, etc., to form a stent or scaffold 150, as shown in the example in the side view of FIG. 15. The stent or scaffold 150 may thus be formed from the coated substrate 130′, in one example, such that the connecting struts 152 are formed from the thickened elastomeric polymer segments 138 while the circumferential segments 154 may be formed from the polymeric substrate 130′. This may result in a contiguous and uniform stent or scaffold structure 150 which maintains high-strength segments 154 connected to one another via elastomeric struts 152 such that structure 150 exhibits high-strength characteristics yet is flexible overall.

In yet another variation, a stent or scaffold 160 structure may be formed from the coated polymeric substrate 130′ such that a first circumferential segment 162 is formed from the elastomeric polymer segments 138 while an adjacent second circumferential segment 164 is formed from substrate 130′ such that second segment 164 is relatively higher in strength than first segment 162, which is relatively more flexible, as shown in the side view of FIG. 16. The alternating segments of elastomeric segments and substrate segments may be repeated along a portion or the entire length of structure 160 depending upon the desired degree of flexibility and strength characteristics. Moreover, other variations of alternating between the segments may be employed, if so desired, as these examples are not intended to be limiting.

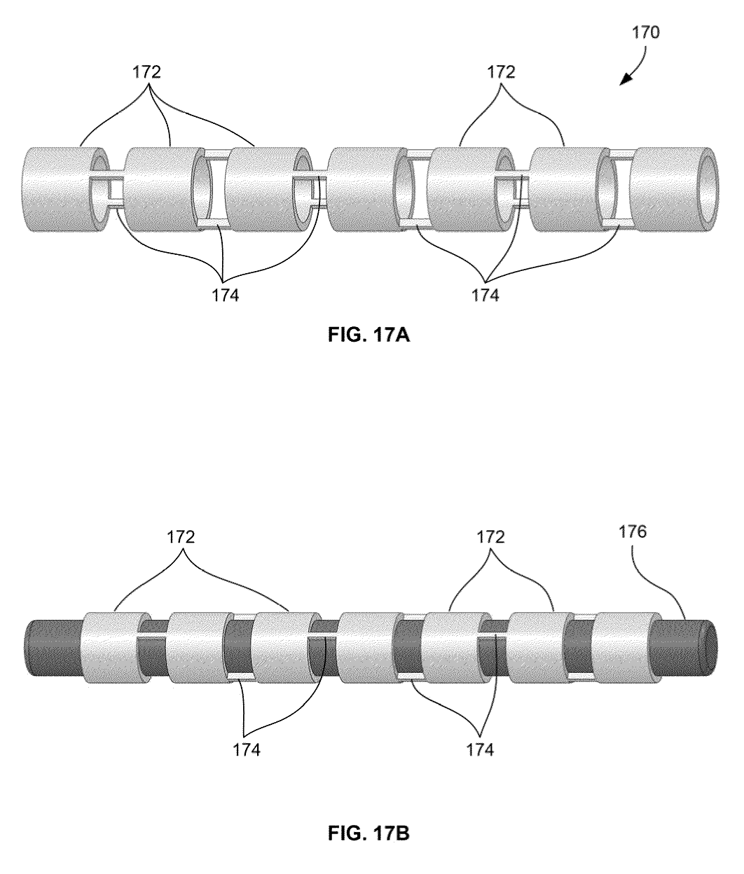

Another variation for fabricating a composite structure is shown in the perspective view of FIG. 17A. A substrate tubing can be formed by dip-coating and the resulting substrate may be machined, as described above, into a substrate 170 having a number of ring segments 172 which are connected via connecting members 174. Although seven ring segments 172 are shown in this example, fewer than or greater than seven ring segments 172 may be utilized and the connecting members 174 may be fashioned into alternating apposed members between adjacent segments 172, as shown, or in any other patterns as practicable. Once the substrate 170 has been desirably machined, substrate 170 may be positioned upon mandrel 176, as shown in FIG. 17B.

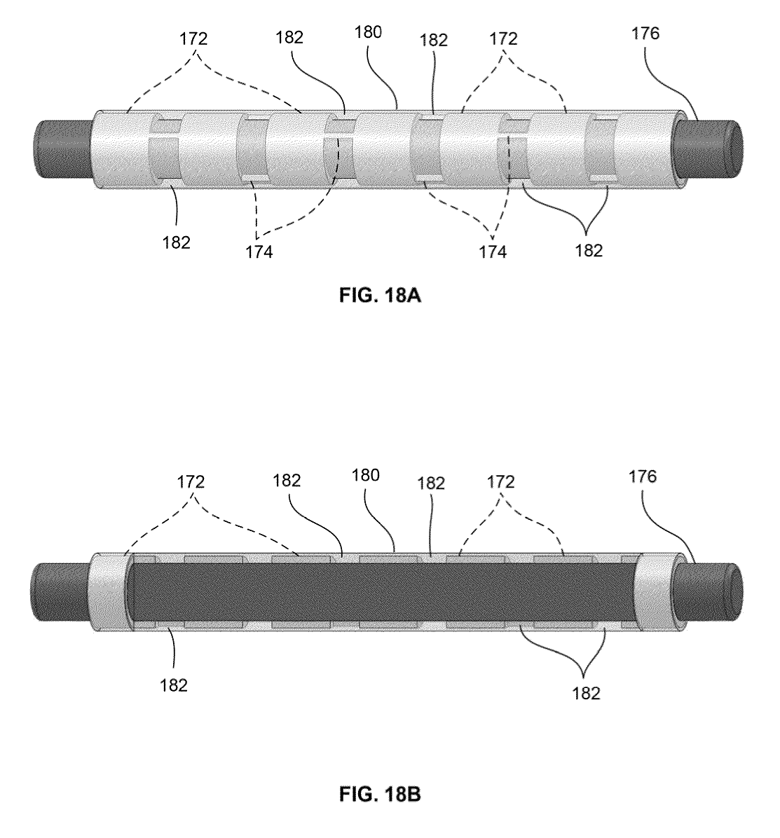

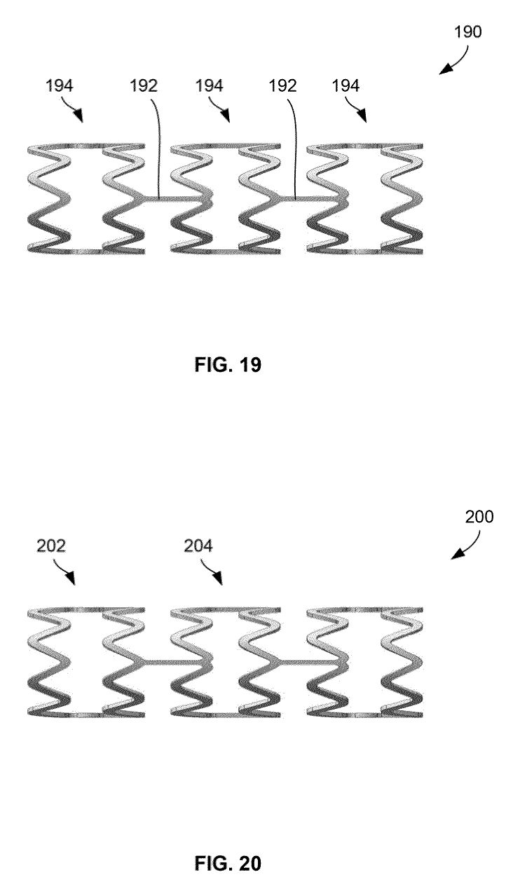

The mandrel 176 and substrate 170 may then be coated again, e.g., via dip-coating as previously described, by one or more layers of bio-absorbable elastomeric polymers 180 which may be coated upon the machined portions to form thickened elastomeric polymer segments 182 as well as upon ring segments 172, as shown in the respective side view and cross-sectional side view of FIGS. 18A and 18B. The use of connecting members 174 between adjacent ring segments 172 may allow for the structure to maintain a high precision axial distance between each of the ring segments 172. The resulting composite structure may be processed and/or machined to form one or more stents or scaffolds having various composite structural characteristics. 100761 An example of such a stent or scaffold 190 is shown in the side view of FIG. 19. In this example, the connecting struts 192 may be formed of the elastomeric polymer from polymer segments 182 while the circumferential segments 194 may be formed from the ring segments 172. The resulting stent or scaffold 190 allows for the structure to have significant flexibility along the axial, torsional, and/or bending directions as well as the ability to withstand relatively long fatigue cycles without formation of cracks or fractures, e.g., 1,000,000 to 3,000,000 cycles, in axial compression, extension, and torsional modes. Also, the stent or scaffold 190 may also withstand a pulsatile fatigue life of up to, e.g., 120,000,000 cycles or more. The connecting members 174 may be utilized as part of either the resulting circumferential segments 194 and/or connecting struts 192, if so desired; otherwise, connecting members 174 may be removed or machined off during the processing of the stent or scaffold 190 leaving only the ring segments 172 and elastomeric polymer segments 182.

In yet another variation, stent or scaffold 200 structure, shown in FIG. 20, may be formed of alternating high strength and elastomeric circumferential segments with elastomeric of non-elastomeric connecting struts. In this example, first circumferential segment 202 may be formed from the elastomeric polymer segments 182 and second circumferential segment 204 may be formed from the ring segments 172 such that alternating elastomeric segments are relatively more flexible to yield a structure which is flexible overall yet still retains high strength and long fatigue life. Each subsequent ring segment may be alternated while the connecting struts may be elastomeric or non-elastomeric or an alternating arrangement of both elastomeric and non-elastomeric struts.

In yet another example, the ring segments may be fabricated to a first diameter and expanded to a larger second diameter using, e.g., a blow molding process. This may be accomplished immediately post dip coating while the ring structures are semi-dry and relatively flexible, e.g., where any residual solvent is greater than 40%. The blow molding process may orient the molecular chains to a circumferential direction to improve the radial strength of the ring segments. Examples of blow molding dip-coated substrates are described in further detail in U.S. patent application Ser. No. 12/143,659, which has been incorporated by reference hereinabove.

The applications of the disclosed invention discussed above are not limited to certain processes, treatments, or placement in certain regions of the body, but may include any number of other processes, treatments, and areas of the body. Modification of the above-described methods and devices for carrying out the invention, and variations of aspects of the invention that are obvious to those of skill in the arts are intended to be within the scope of this disclosure. Moreover, various combinations of aspects between examples are also contemplated and are considered to be within the scope of this disclosure as well.

What is claimed is:

1. A composite substrate for forming a stent structure, comprising:
a tubular polymeric substrate having one or more segments reduced in diameter defined along a length of the substrate; and,
at least one layer of an elastomeric polymer coating laid atop an outer surface of the polymeric substrate such that the elastomeric polymer is contained within the one or more reduced segments to form elastomeric polymer segments.

2. The substrate of claim 1 wherein the tubular polymeric substrate is formed via a dip-coating process.

3. The substrate of claim 1 wherein the one or more reduced segments are uniformly spaced apart from one another.

4. The substrate of claim 1 wherein the at least one layer forms a uniform diameter upon the outer surface of the polymeric substrate.

5. The substrate of claim 1 further comprising additional layers of an elastomeric polymer coating laid atop the at least one layer.

6. The substrate of claim 1 wherein the one or more reduced segments are reduced through the substrate such that the substrate forms a plurality of ring segments.

7. The substrate of claim 6 wherein adjacent ring segments are connected via at least one connecting member formed from the polymeric substrate.

8. A composite stent structure, comprising:
a first circumferential segment comprised of an elastomeric polymer;
at least a second circumferential segment comprised of a non-elastomeric polymer substrate; and
at least one connecting strut coupling the first and second circumferential segments such that the stent structure forms a contiguous and uniform structure.

9. The stent structure of claim 8 wherein the first circumferential segment comprises an expandable stent ring segment.

10. The stent structure of claim 8 wherein the second circumferential segment comprises an expandable stent ring segment.

11. The stent structure of claim 8 wherein the first circumferential segment is formed from an elastomeric polymer segment formed on a tubular polymeric substrate having one or more segments reduced in diameter defined along a length of the substrate.

12. The stent structure of claim 11 wherein the second circumferential segment is formed from the tubular polymeric substrate.

13. The stent structure of claim 8 further comprising additional circumferential segments connected to an adjacent segment via at least one connecting strut.

14. The stent structure of claim 13 wherein the additional circumferential segments alternate between the elastomeric polymer and the non-elastomeric polymer.

15. The stent structure of claim 13 wherein the additional circumferential segments are connected via the at least one connected strut which is comprised of the elastomeric polymer.

16. A method for forming a composite stent structure, comprising:
processing a polymeric tubular substrate such that one or more segments are reduced in diameter along a length of the substrate between corresponding one or more ring segments;
coating an elastomeric polymer upon an outer surface of the tubular substrate such that the elastomeric polymer is contained within the one or more reduced segments to form elastomeric polymer segments; and
further processing the tubular substrate to form a stent structure having at least a first circumferential segment formed from the elastomeric polymer segment and at least a second circumferential segment formed from the polymeric tubular substrate, at least one connecting strut coupling the first and second circumferential segments such that the stent structure forms a contiguous and uniform structure.

17. The method of claim 16 further comprising forming the polymeric tubular substrate via dip-coating.

18. The method of claim 16 wherein processing a polymeric tubular substrate comprises removing the diameter along the one or more reduced segments.

19. The method of claim 16 wherein processing a polymeric tubular substrate comprises forming at least one connecting member along the reduced segments between each of the one or more ring segments.

20. The method of claim 16 wherein coating an elastomeric polymer comprises dip-coating the elastomeric polymer upon the outer surface.

21. The method of claim 16 wherein coating an elastomeric polymer comprises forming at least one coat of the elastomeric polymer such that a uniform diameter is formed along the tubular substrate.

22. The method of claim 16 wherein further processing the tubular substrate comprises forming additional circumferential segments connected via at least one connecting strut between adjacent segments.

23. The method of claim 22 wherein the additional circumferential segments alternate between the elastomeric polymer segment and the polymeric tubular substrate.

24. The method of claim 22 wherein the additional circumferential segments are connected via the at least one connecting strut which is comprised of the elastomeric polymer.

25. A composite stent structure, comprising:
a base polymeric layer;
one or more ring structures having a formed first diameter and being separated from one another and positioned axially upon the base polymeric layer, the one or more ring structures being radially compressible to a smaller second diameter and re-expansion to the first diameter;
an overlaid polymeric layer formed atop the base polymeric layer and the one or more ring structures,
wherein the ring structures are encased between the base and overlaid polymeric layers and are coupled to one another via segments of the base and overlaid polymeric layer such that adjacent ring structures are axially and rotationally movable relative to one another and where the one or more ring structures are configured to be formed into a scaffold structure.

26. The stent structure of claim 25 wherein the base polymeric layer and overlaid polymeric layer are elastomeric.

27. The stent structure of claim 25 wherein the one or more ring structures are radially deformable.

28. The stent structure of claim 25 wherein the base polymeric layer and the overlaid polymeric layer are fabricated from a common polymer.

29. The stent structure of claim 25 wherein the base polymeric layer and the overlaid polymeric layer are fabricated from different polymers.

30. The stent structure of claim 25 wherein the one or more ring structures are uniformly spaced from one another.

31. The stent structure of claim 25 wherein the one or more ring structures are spaced closer to one another along a first portion than along a second portion of the stent structure.

32. The stent structure of claim 25 wherein a terminal ring structure is relatively more flexible than a remainder of the ring structures.

33. The stent structure of claim 25 wherein alternating ring structures are fabricated from different polymers.

34. The stent structure of claim 25 wherein the ring structure comprises a helical member.

35. The stent structure of claim 25 wherein the one or more ring structures are each fabricated from different polymers.

36. The stent structure of claim 25 wherein the one or more ring structures each have a width ranging from 1 mm to 10 mm.

37. The stent structure of claim 25 wherein the one or more ring structures are separated from one another by 1 mm to 10 mm.

38. A method of forming a composite stent structure, comprising:
forming a base polymeric layer upon a mandrel;
overlaying one or more ring structures upon the base polymeric layer such that the ring structures are separated from one another and positioned axially thereupon;
forming an overlaid polymeric layer atop the base polymeric layer and the one or more ring structures; and
forming the one or more ring structures into scaffold structures such that surfaces of the one or more ring structures are exposed from the base and overlaid polymeric layers.

39. The method of claim 38 wherein forming a base polymeric layer comprises forming an elastomeric bioabsorbable layer upon the mandrel.

40. The method of claim 38 wherein overlaying comprises providing a high strength polymeric substrate machined to form the one or more ring structures.

41. The method of claim 38 wherein overlaying comprises positioning the one or more rings at a distance of 1 mm to 10 mm from one another.

42. The method of claim 38 wherein forming an overlaid polymeric layer comprises forming an elastomeric bioabsorbable layer upon the base polymeric layer and the one or more ring structures.

43. The method of claim 38 wherein forming the one or more ring structures machining the structures to expose the surfaces.

44. The method of claim 38 further comprising radially compressing the composite stent structure from a first formed diameter to a second delivery diameter which is smaller than the first diameter.
